(19) United States
(12) Patent Application Publication
(10) Pub. No.: US 2021/0330412 A1
(43) Pub. Date: Oct. 28, 2021

(54) Medical Draping Apparatus

(71) Applicant: Thomas Zeigler, Windber, PA (US)

(72) Inventor: Thomas Zeigler, Windber, PA (US)

(21) Appl. No.: 16/899,680

(22) Filed: Jun. 12, 2020

Related U.S. Application Data
(60) Provisional application No. 63/016,775, filed on Apr. 28, 2020.

(51) Int. Cl. A61B 46/20; A61B 90/50

(52) U.S. Cl. CPC A61B 46/20; A61B 90/50

(57) ABSTRACT

A draping apparatus for providing a medical procedure barrier. The draping apparatus comprises a generally transparent medical draping structure for use during diagnostic, therapeutic, and surgical procedures performed on a patient. The draping structure is supported by a support structure that is removably attachable to a bed. An access component comprises a pair of long armed procedure gloves affixed to the draping structure that allows a medical provider to insert their hands into the gloves to provide assistance to the patient without exposure to aerosol contaminants. The invention enables medical procedures, such as positive pressure ventilation and endotracheal intubation, to be performed from behind the protective draping apparatus. The invention also allows for quick and easy disposal, with minimal need for cleaning.

### CROSS-REFERENCE TO RELATED APPLICATION

The present application claims priority to, and the benefit of, U.S. Provisional Application No. 63/016,775, which was filed on Apr. 28, 2020 and is incorporated herein by reference in its entirety.

### BACKGROUND

The present invention generally relates to protective drapes, and more specifically to a draping apparatus designed to enable a medical professional to provide treatment to a patient without the medical professional and/or patient being exposed to potential contaminants such as, without limitation, aerosol contaminants, coughs, secretions, and the like. Accordingly, the present specification makes specific reference thereto. However, it is to be appreciated that aspects of the present invention are also equally amenable to other like applications, devices and methods of manufacture.

Hospitals, physicians, and other healthcare workers are increasingly concerned about the incidence of secondary infection as well as transmission of communicable diseases from a patient to health care facility providers (and vice versa) occurring during medical procedures. As a result, more attention is being given to the establishment and maintenance of protective or sterile fields around infected patients and procedure sites. For example, some healthcare facilities require medical professionals to check certain conditions, such as whether a proper sterile field has been established or whether a proper sterile field can be maintained around a patient, before proceeding with a medical procedure. Despite these requirements, and given the demands currently being placed on medical professionals, it can sometimes be difficult to remember to check each precondition for a sterile environment before proceeding. Further, it can also be difficult to maintain sterile fields with the currently available equipment and sterilization methods. As a result, communicable and infectious diseases may spread from the patient to healthcare workers such, as doctors, nurses, other caretakers, etc. or vice versa at an accelerated rate.

A medical drape is one device used to combat the potential transmission of diseases or other contaminants during a medical procedure. More specifically, medical or surgical drapes may be used to cover a patient during the performance of surgical or other medical procedures, and therefore help to prevent the possibility of infection or contaminants being transmitted from the patient to the medical staff or caregivers, or vice versa. Medical drapes provide protection to the medical staff while taking care of a patient by creating and maintaining an effective barrier that minimizes the transmission of microorganisms between non-sterile and sterile areas. To perform adequately, the drape should be made of a medical grade material that is resistant to blood, plasma, serums, and/or other bodily fluids in an effort to reduce the likelihood that such fluids could contaminate an otherwise sterile environment. In general, such drapes include a sheet provided with an opening or fenestration through which a caregiver can gain access to a patient.

As noted, existing medical or protective drapes typically include fenestrations or openings at different positions to allow access to the patient. However, not all fenestrations may be needed or utilized during each medical procedure, and the unused fenestrations are unnecessary exposure pathways to the medical procedure, which may lead to the transmission of microorganisms from the exposed area of the patient's body to the medical professional and/or worker. Stated differently, while the openings in the protective drape are useful for allowing the doctors and nurses to insert their hands through the protective drape to perform the procedure or otherwise treat the patient, said openings may also facilitate cross-contamination and the spread of diseases from patients to medical or health workers, and vice versa.

Additionally, most medical or protective drapes are configured to accommodate a particular medical or surgical procedure. Therefore, medical facilities, such as hospitals, ambulatory surgical centers, and the like, typically stock multiple drapes of different sizes and shapes in order to fulfil the specific needs of doctors and other medical staff. This leads to facilities having to procure and maintain a relatively large number of medical drapes and medical equipment, which not only increase costs and the amount of space needed to store the same, but it also unnecessarily increases the complexity of the facility's inventory management system.

Furthermore, the existing medical drapes used for protection in pandemics for communicable diseases are not portable and remain fixed once the drape is set over the patient in a desired position. Also, the size of the medical drape is not adjustable, thereby making it difficult for the medical staff to manage multiple numbers of drapes. Moreover, when the medical staff must set up different drapes of different sizes to perform different surgical procedures on the same patient, there is a greater likelihood of noncompliance and/or mistake which in turn results in increased risk to the medical providers and/or the patient.

Therefore, there exists a long felt need in the art for a medical drape that completely prevents cross-contamination by contact with other infected persons, objects and air. More specifically, there is a long felt need in the art for a protective medical drape which can protect medical or health workers from the spread of infection, while performing surgical and other medical procedures. There is also a long felt need in the art for a protective drape that has no open areas such as open holes or open fenestrations which minimizes the infection spread between patients and medical workers, and for a medical drape that serves multiple purposes and is of configurable size. Additionally, there is a long felt need in the art for a medical drape that is adjustable in size and that is portable. Finally, there is a long felt need in the art for prepackaged surgical drapes which are compact, easy to open, and allow ready access to a refolded drape, thereby minimizing the dangers of contamination during removal from the package and subsequent unfolding and draping.

The present invention, in one exemplary embodiment, is a draping apparatus which is designed to enable a medical professional to provide treatment to an infected patient without being exposed to aerosol contaminants, patient coughs, patient secretions, and the like. The draping apparatus can also be utilized in other industries where protection from contaminates is needed. The draping apparatus provides a way for a healthcare provider to inspect the patient without direct physical touching. The draping apparatus of the present invention may be manufactured in various sizes to suit user need and/or preference. Further, the protective drape is portable and is relatively easy to setup.

In this manner, the improved drape of the present invention accomplishes all of the forgoing objectives, thereby providing an easy solution to prevent contamination of medical staff and physicians from patient discharges and vice versa. A primary feature of the present invention allows physicians to inspect a patient without actually touching the patient, thereby reducing the risk of contamination. Finally, the improved draping apparatus of the present invention is capable of improving the sterile environment around an ill patient, while also protecting the medical staff and physicians.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, comprises a draping apparatus which is designed to enable a medical professional to provide treatment to an ill or infected patient without being exposed to aerosol contaminants, patient coughs, patient secretions and other contaminants. The draping apparatus comprises extendable gloves that can be used by a physician to inspect a patient, without touching the patient directly, and that can be affixed at any location depending on need or preference.

More specifically, the draping apparatus comprises a support structure, a draping structure, and an access component. The draping apparatus is configured to provide a protective barrier for performing medical procedures with a relatively low risk of exposure to contamination. The draping structure is adjustably attached to the support structure which is, in turn, attachable to a bed. Once attached to the bed, the draping structure at least partially encapsulates the bed, thereby forming a protective barrier therearound. The access component of the invention may then be integrated into the draping structure so that a healthcare provider can safely access an interior of the draping structure to attend to the patient.

In one embodiment, the support structure comprises a plurality of vertical support poles, a plurality of clamping elements, and a plurality of adjusting elements. The vertical support poles attach to or independently support the draping structure around the patient's bed. Further, the plurality of clamping elements attach the draping structure to the vertical support poles. The plurality of adjusting elements allow for adjustment in height of the draping structure over the bed to suit user need and/or preference.

In a preferred embodiment, the draping structure comprises a plurality of transparent surfaces connected by a plurality of seams and a plurality of corners configured to form a general box shape around the bed. The access component comprises a pair of procedure gloves penetrating one of the transparent surfaces closest to the head of the bed. The pair of procedure gloves are attached to the transparent surface closest to the head through a pair of glove holes or openings. The access component may further comprise a pair of inner glove retaining elements. The pair of inner glove retaining elements are configured to integrate each procedure glove into the draping structure at the respective glove hole or opening to form an airtight connection.

In a further preferred embodiment, the draping apparatus may also comprise an equipment access port. The equipment access port may be configured to permit access of medical equipment wiring and tubing through a partially sealable opening to in the drape to limit exposure. The draping apparatus may further comprise an air exchange component that is comprised of an air filter, an air inlet and an air outlet, as described more fully below.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the draping apparatus of the present invention substantially encapsulating a patient in a bed in accordance with the disclosed architecture.

FIG. 2 illustrates a front perspective view of one potential embodiment of the draping apparatus of the present invention and illustrating a pair of gloves in accordance with the disclosed architecture.

FIG. 3 illustrates a side perspective view of one potential embodiment of the draping apparatus of the present invention and illustrating a medical provider attending to a patient without direct contact in accordance with the disclosed architecture.

FIG. 4 illustrates a side perspective view of an alternative embodiment of the draping apparatus of the present invention further comprising a filter and an equipment access port in accordance with the disclosed architecture.

FIG. 5 illustrates a perspective view of an alternative embodiment of the draping apparatus of the present invention further comprising an air exchange component in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They do not intend as an exhaustive description of the invention or do not limit the scope of the invention. Additionally, an illustrated embodiment need not have all the aspects or advantages shown. Thus, in other embodiments, any of the features described herein from different embodiments may be combined.

The present invention in one exemplary embodiment is a medical drape for use during diagnostic, therapeutic, and surgical procedures performed on an ill or infected patient. The medical drape is comprised of a pair of corresponding and long armed procedure gloves that are affixed to the drape. The presence of the gloves allow a medical provider, such as a physician, nurse or other medical staff, to insert his or her hands into the gloves to provide assistance to the patient on the other side of the drape without exposure to aerosol contaminants, patient coughs, secretions, etc. The invention also enables procedures, such as positive pressure ventilation and endotracheal intubation, to be performed from behind the protective drape. Advantageously, the medical drape of the present invention provides for improved installation of a barrier, improved visibility of a patient, and inspecting the patient without transmission of any patient fluid between sterile and non-sterile areas while also allowing for freedom of movement of the hands of a physician or other healthcare provider via the protective gloves.

Additionally, it is contemplated that the drape of the present invention can also be utilized in other industries where workers may otherwise be exposed to harmful droplets, contaminants, fumes, etc. Accordingly, the present invention benefits any industry that desires a working drape with affixed gloves to create a more sterile environment, such as sanding, painting, laboratories, and the like. The present invention further allows for quick and easy disposal, with minimal need for cleaning.

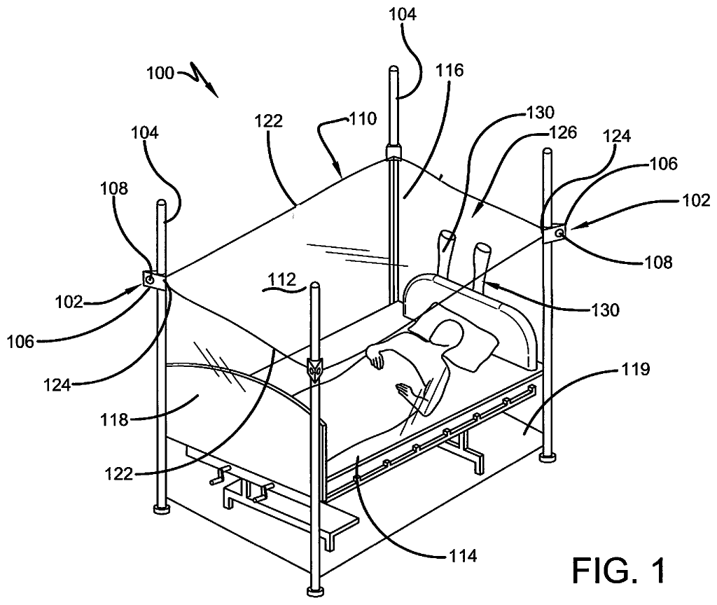

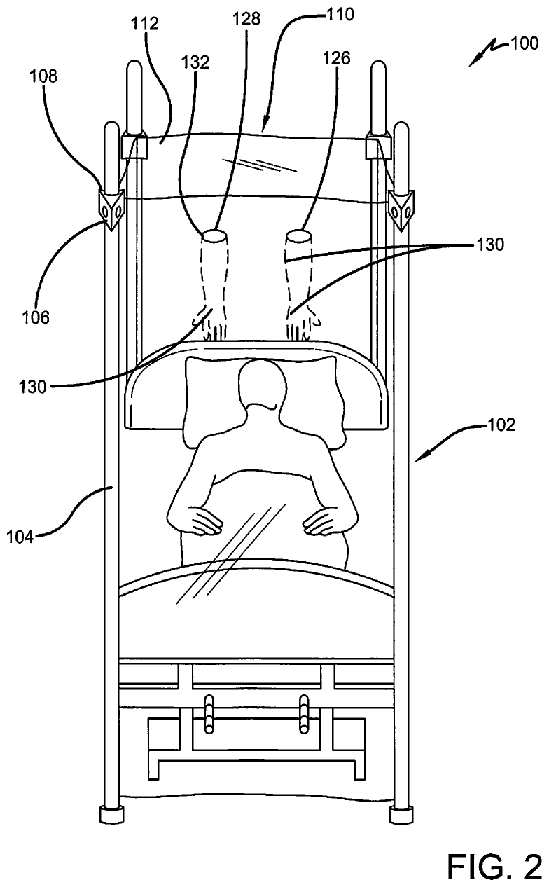

Referring initially to the drawings, FIG. 1 illustrates a perspective view of one potential embodiment of the draping apparatus 100 of the present invention substantially encapsulating a patient in a bed in accordance with the disclosed architecture, and FIG. 2 illustrates a front perspective view of the draping apparatus 100 of the present invention and comprising a pair of procedure gloves 130, which are described below in greater detail. More specifically, the draping apparatus 100 comprises a support structure 102, a draping structure 110, and an access component 126. The draping apparatus 100 is configured to provide a protective barrier for healthcare providers while performing medical procedures, and reduces the risk of exposure to contamination to or from the patient. The draping structure 110 is adjustably attached to the support structure 102, which is removably attachable to a bed, cot, or procedure table. Once attached to the bed, the draping structure 110 at least partially encapsulates the bed, thereby forming the protective barrier. As explained more fully below, the access component 126 is integrated into the draping structure 110 so that a healthcare provider can safely access an interior of the draping structure 110.

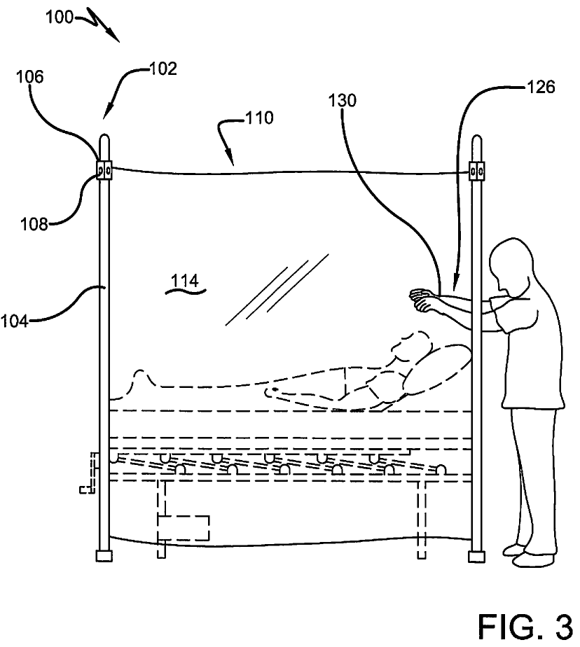

In a preferred embodiment of the present invention, the support structure 102 comprises a plurality of vertical support poles 104, a plurality of clamping elements 106, and a plurality of adjusting elements 108. The vertical support poles 104 attach to or independently support the draping structure 110 around the bed, and the plurality of clamping elements 106 attaches the draping structure 110 to the vertical support poles 140, as best shown in FIGS. 1 through 3. The plurality of adjusting elements 108 allows for adjustment of the draping structure 110 over the bed.

The draping structure 110 further comprises a plurality of surfaces connected by a plurality of seams 122 and a plurality of corners 124 configured to form a generally box or rectangular shape around the bed. The plurality of surfaces may comprise a drape top surface 112, a pair of drape side surfaces 114, a drape head side surface 116, a drape foot side surface 118, and a drape bottom surface 119, as best shown in FIG. 1. Each of the plurality of surfaces is preferably transparent or translucent.

Additionally, the draping structure 110 is preferably manufactured from a transparent two millimeter poly plastic with dimensions 8 feet wide by 8 feet long. An advantage of these dimensions is that the draping apparatus 100 is relatively large, thereby ensuring an unobstructed patient view in a wide range of applications. Further, the transparent poly plastic enables better site monitoring, patient monitoring and better positioning. In use, the draping structure 110 may be unfolded or fixed such that the drape head side surface 116 of the draping structure 110 is placed over a patient's head. The draping structure 110 size and material may vary as desired or needed to suit user need or preference. For example, the draping apparatus dimensions could be smaller for pediatric patients. Additionally, the material of manufacture may be any plastic material as long as it is relatively transparent and capable of being decontaminated or easily disposed of

The draping apparatus 100 can be set up around a patient bed using the plurality of vertical support poles 104. The vertical support poles 104 may be either attached to a patient's bed or be free standing on the ground with the help of stands. Additionally, the vertical support poles 104 may be fixed, or may have wheels and an associated brake on the stand bottom for easy repositioning and in order to set up the draping apparatus 100. The plurality of clamping elements 106 and the plurality of adjusting elements 108, such as adjusting knobs, engage each vertical support pole 104 to secure the draping structure 110 from its four corners 124. The clamping elements 106 can also be used to adjust the height of the draping structure 110 with respect to the patient. The clamping elements 106 themselves may be designed to be capable of repeated securing and un-securing events without failure. However, it is contemplated that other securing elements can also be used to secure and fix the draping structure 110 to the vertical support poles 104. Examples of such alternative clamping elements 106 may include, but are not limited to, snaps, screws, hook and loop fasteners, and clips. Alternatively, the clamping elements 106 may be designed for temporary securing and un-securing events.

The access component 126 of the present invention comprises a pair of procedure gloves 130 extending inwardly (i.e., in the direction of the patient) from the draping structure 110. The pair of procedure gloves 130 may be integrally formed into the drape head side surface 116, or other suitable location. Alternatively, the pair of procedure gloves 130 may be attached to the drape head side surface 116 through a pair of glove holes or openings 128. The access component 126 may further comprise a pair of inner glove retaining elements 132. The pair of inner glove retaining elements 132 is configured to integrate each procedure glove 128 into the draping structure 110 at the respective glove opening 128, thereby forming an airtight connection.

However, any number of the glove openings 128 and procedure gloves 130 can be present on any of the plurality of surfaces of the draping structure 110, and the procedure gloves 130 can be affixed on any side surface of draping structure 110 as per the needs or wants of the particular user. In one embodiment illustrated in FIG. 2, the pair of long-armed two millimeter thick plastic gloves 130 are integrated into the drape top surface 112. The procedure gloves 130 are preferably placed approximately one foot from either side of a midline 120 in an anatomical position, and to provide maximum access to the patient.

The draping structure 110 may be configured so as to releasably or permanently retain the pair of procedure gloves 130, which further comprise a right-hand glove and a left-hand glove. In such an embodiment, the draping structure 110, from an outer surface (i.e. the side of the surface opposite the patient) incorporates the pair of glove holes 128 shaped to receive hands of the physician or the medical staff. The inner glove retaining elements 132 are positioned around each of the openings 128 of the procedure gloves 130 at a joint portion with the draping structure 110. The inner glove retaining elements 132 may be a thick ring-shaped structure or lining, to strongly affix the procedure gloves 130 to the draping structure 110 adjacent to openings 128. In such an embodiment, the draping structure 110 may be provided with repeated arrays of the features described above which enable multiple gloves 130 or multiple pairs of gloves 130 to be releasably or permanently retained thereby.

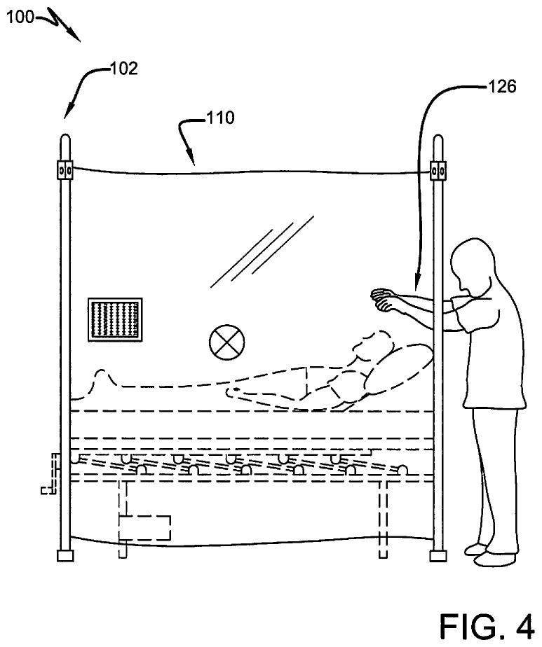

As best illustrated in FIG. 4, the draping apparatus 100 may further comprise an equipment access port 134. The equipment access port 134 is configured to permit access of medical equipment wiring and tubing through a partially sealable opening in one of the plurality of surfaces of the draping structure 110 to limit exposure and reduce the likelihood of contamination. Typically, the equipment access port 134 would be integrated into a different one of the transparent surfaces of the draping structure 110. For example, if the access component 126 was integrated into the drape head side surface 116, the equipment access port 134 could be integrated into one of the drape pair of side surfaces 114.

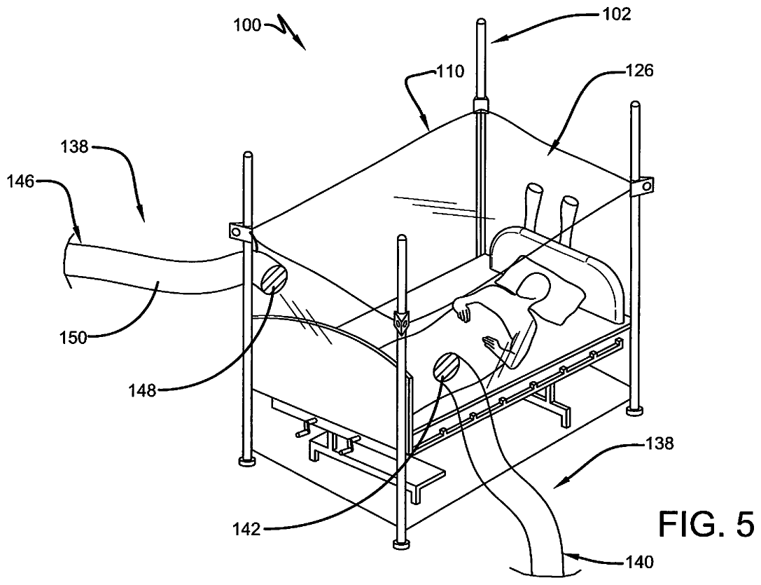

As best illustrated in FIGS. 4 and 5, the draping apparatus 100 may further comprise an air exchange component 138 for providing fresh air and removing stale air from the enclosure. The air exchange component 138 may comprise an air filter 136, such as a N-95 or HEPA filter. Alternatively, or in addition thereto, the air exchange component 138 may comprise an air inlet 140 and an air outlet 146. The air inlet 140 may comprise an air tube 144 integrated into an inlet opening 142 in one of the plurality of surfaces of the draping structure 110. The air outlet 148 may comprise an air tube 150 integrated into an outlet opening 148 in one of the plurality of surfaces of the draping structure 110. As such, the draping structure 110 could be slightly pressurized on the inside.

The draping apparatus 100 enables procedures, such as positive pressure ventilation and endotracheal intubation, to be performed from outside the draping structure 110. The transparent draping structure 110 enables physicians to place a medical device such as an endotracheal tube into the windpipe (trachea) through the mouth or nose without contamination. Further, a device such as a mechanical ventilator is easily placed by the physician using the gloves 130 affixed to the draping structure 110 on a critically ill patient. It would be apparent to anyone skilled in the art, that the draping structure 110 benefits any other industry that needs a draping apparatus with affixed gloves to provide a more sterile environment to a user. The present invention allows for quick and easy disposal with minimal need for cleaning.

For a medical procedure, for example, a patient may be positioned on the patient bed. The draping structure 110 of the present invention may then be placed over the patient and around the perimeter of the bed. In a preferred embodiment, the procedure gloves 130 are positioned along the drape head side surface 116 to enable the doctors, medical staff, nurses, etc. to better access the patient during various different type of medical procedures, such as positive pressure ventilation and endotracheal intubation. In the most preferred embodiments, the physician or other medical staff can insert his or her hands into the procedure gloves 130 to inspect the patient, thereby creating a sterile field around them.

The draping apparatus 100 of the present invention can be manufactured using any suitable medical grade materials. A material of the drape is preferably selected to create and maintain an effective barrier layer between a patient and medical staff, such that the material minimizes the passage of contaminants such as cough droplets, microorganisms, etc. between non-sterile area around the patient, and sterile areas around the physician. To be effective, the barrier material of the draping structure 110 should be resistant to blood, aqueous fluid, and abrasions, and as lint-free as possible. During medical operations and for intraoperative patient, the draping structure 110 prevents blood and other bodily fluids and secretions from contaminating the physicians present in the sterile field.

In an embodiment of the present invention, the draping structure 110 is made up of any suitable material selected from, but not limited, to disposable nonwoven fabrics, plastic films, or papers. In alternative embodiments, the draping structure 110 may be of potentially suitable materials include polymeric (plastic) sheet materials, e.g., polyethylene (PE), polyvinylchloride (PVC), polypropylene (PP), and combinations or mixtures thereof. In a further preferred embodiment, the material may be a biocompatible medical grade plastic which may be completely transparent or substantially transparent. The material of the draping structure 110 may allow air circulation while maintaining a barrier to fluid and microorganisms.

In an embodiment, the draping structure 110 may be fabricated from a material that is capable of being sterilized by gas or gamma-sterilization techniques commonly used in hospitals and other medical facilities. Examples of gas-sterilization techniques usable with the various embodiments discussed herein, include those using Ethylene Oxide (EtOH) gas, e.g., those known as gas diffusion sterilization techniques. Further, the draping structure 110 should be resistant to punctures and tears to prevent microbial contamination. The material may be also designed to reduce glare from operating lights.

There are virtually innumerable uses for the present invention, all of which need not be detailed here. For instance, the draping apparatus 100 could be used in any setting where sterility concerns exist. Furthermore, the draping apparatus 100 could be used in other contexts to shelter an environment beneath the draping apparatus 100. All the disclosed embodiments can be practiced without undue experimentation. In non-medical use, the draping apparatus 100 of the present invention can be used generally to prevent non-impact exposure to liquid splash and dust during the course of activities such as but not limited to, painting, automotive repairing, and general assembly line work where dust and spray are likely to occur and can affect the health of the user. The draping apparatus 100 of the present invention can be used as an improved face shield apparatus providing improved protection for the user's face from splashes of bodily fluids and other foreign materials.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “physician”, “medical professional”, “doctors”, “nurses” and “medical staff” are interchangeable and refer to a person involved in the inspection of a critically ill patient inside the draping structure 110 of the present invention.

It is contemplated that the draping apparatus 100 constructed in accordance with the present invention will be tailored and adjusted by those of ordinary skill in the art to accommodate various levels of performance demand imparted during actual use. Accordingly, while this invention has been described by reference to certain specific embodiments and examples, it will be understood that this invention is capable of further modifications. This application is, therefore, intended to cover any variations, uses or adaptations of the invention following the general principles thereof, and including such departures from the present disclosure as come within known or customary practice in the art to which this invention pertains and fall within the limits of the appended claims.

Notwithstanding the forgoing, the draping apparatus 100 of the present invention and its various structural components can be of any suitable size, shape, and configuration as is known in the art without affecting the overall concept of the invention, provided that it accomplishes the above stated objectives. One of ordinary skill in the art will appreciate that the shape and size of the draping apparatus 100 and its various components and material, as shown in the FIGS. are for illustrative purposes only, and that many other shapes and sizes of the draping apparatus 100 are well within the scope of the present disclosure. Although the dimensions of the drape 100 are important design parameters for attaining maximum sterile environment, the draping apparatus 100 and its components may be of any shape or size that ensures optimal performance during use and/or that suits user need and/or preference.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A draping apparatus comprising:
a support structure;
a draping structure adjustably attached to the support structure; and
an access component integrated into the draping structure.

2. The draping apparatus of claim 1, wherein the support structure is attachable to a bed so that the draping structure at least partially encapsulates the bed.

3. The draping apparatus of claim 1, wherein the support structure comprises a plurality of vertical support poles, a plurality of clamping elements for attaching the draping structure to each vertical support pole, and a plurality of adjusting elements.

4. The draping apparatus of claim 1, wherein the draping structure comprises a plurality of surfaces connected by a plurality of seams.

5. The draping apparatus of claim 1, wherein the access component comprises a pair of procedure gloves integrated into the draping structure.

6. The draping apparatus of claim 5, wherein the integration of the pair of procedure gloves into the draping structure is airtight.

7. The draping apparatus of claim 1, wherein the draping structure is transparent.

8. A draping apparatus for providing a medical procedure barrier, the draping apparatus comprising:
a support structure attachable to a bed;
a draping structure comprising a plurality of transparent surfaces adjustably attached to the support structure; and
an access component integrated into one of the transparent surfaces of the draping structure.

9. The draping apparatus of claim 8, wherein the support structure comprises a plurality of vertical support poles, a plurality of clamping elements for attaching the draping structure to each vertical support pole, and a plurality of adjusting elements.

10. The draping apparatus of claim 8, wherein the plurality of transparent surfaces are connected by a plurality of seams and a plurality of corners.

11. The draping apparatus of claim 8, wherein the draping structure is adjustable in height by the support structure.

12. The draping apparatus of claim 8, wherein the access component comprises a pair of procedure gloves and a pair of inner glove retaining elements for integrating each procedure glove into the draping structure.

13. The draping apparatus of claim 12, wherein the integration of the pair of procedure gloves into the draping structure is airtight.

14. The draping apparatus of claim 8 further comprising an equipment access port integrated into one of the transparent surfaces of the draping structure.

15. The draping apparatus of claim 14, wherein the equipment access port is integrated into a different one of the transparent surfaces of the draping structure than the access component.

16. A draping apparatus for providing a medical procedure barrier, the draping apparatus comprising:
a support structure attachable to a bed;
a draping structure comprising a plurality of surfaces adjustably attached to the support structure;
an access component integrated into the draping structure; and
an air exchange component.

17. The draping apparatus of claim 16, wherein the air exchange component is an air filter.

18. The draping apparatus of claim 16, wherein the air exchange component comprises an air inlet and an air outlet.

19. The draping apparatus of claim 16 further comprising an equipment access port integrated into one of the transparent surfaces of the draping structure.

20. The draping apparatus of claim 16, wherein the draping structure can be pressurized.
